(19) United States
(12) Patent Application Publication
(10) Pub. No.: US 2020/0129838 A1
(43) Pub. Date: Apr. 30, 2020

(54) TRAINING METHOD, TRAINING SYSTEM AND NON-TRANSITORY COMPUTER-READABLE MEDIUM

(71) Applicant: INSTITUTE FOR INFORMATION INDUSTRY, Taipei (TW)

(72) Inventors: Yong-Ruei CHEN, Taipei City (TW); Ping-Che YANG, Taipei City (TW); Shih-Chun CHOU, Taipei City (TW)

(21) Appl. No.: 16/202,111

(22) Filed: Nov. 28, 2018

(30) Foreign Application Priority Data
Oct. 25, 2018 (TW) 107137823

(51) Int. Cl. A63B 71/06; A63B 24/00; A63B 69/00; A61B 5/00; A61B 5/0205; G10L 13/04; G10L 15/22; G10L 15/18

(52) U.S. Cl. CPC A63B 71/0622; A61B 5/024; A63B 24/0062; A63B 69/0028; A61B 5/741; A61B 5/749; A61B 5/0205; G10L 13/043; G10L 15/22; G10L 15/1815; A63B 2071/063; A63B 2230/062; A63B 2230/045; A63B 2230/605; A63B 2220/803; A63B 2230/625; A63B 2069/0037; A63B 2024/0065; A63B 2024/0068; A63B 2220/808; A63B 24/0075

(57) ABSTRACT

A training method, training system and non-transitory computer-readable medium are provided in this disclosure. The training method includes the following operations: receiving a training script; wherein the training script includes at least one training stage corresponding to a timeline, and a training time and a training goal corresponding to the least one training stage; generating a voice information according to the training script, and outputting the voice information by an audio outputting unit; receiving a physiological signal sensed by a physiological signal sensor in the at least one training stage, and analyzing the physiological signal utilized an analyzing module to obtain an analysis result; and determining whether the analysis result meets the training goal corresponding to the at least one training stage; if not, outputting a voice reminding by the audio outputting unit.

### CROSS-REFERENCE TO RELATED APPLICATION

This application claims priority to Taiwanese Application Serial Number 107137823, filed on Oct. 25, 2018, which is herein incorporated by reference.

### BACKGROUND

### Field of Invention

The present application relates to a training method, training system and non-transitory computer-readable medium. More particularly, the present application relates to a training method for automatically generating the training script, training system and non-transitory computer-readable medium.

### Description of Related Art

With the improvement of living standards and health awareness, people began to pay attention to fitness. When the fitness enthusiasts do exercise, the wrong posture will cause certain physical damage; thus the professionals (e.g. fitness coach) are required to provide professional teaching or counseling to the fitness enthusiasts. However, it is quite difficult to hire a fitness coach to follow the fitness enthusiasts at any time, and the cost of fitness coach may make most the fitness enthusiasts discouraged. Therefore, there are many virtual coaching apps on the market. However, the apps only provide course information, detect and record physiological information, and the apps cannot automatically generate the course and the training script suitable the fitness enthusiasts, not only cannot make the virtual coaches interact with the fitness enthusiasts in real time, but also cannot monitor the posture and health conditions of the fitness enthusiasts. Therefore, a training method for automatically generating the training script and interacting with the fitness enthusiasts in real time is required.

### SUMMARY

An aspect of the disclosure is to provide a training method, applied to an electronic device and a server. The training method includes operations of: receiving a training script; wherein the training script includes at least one training stage corresponding to a timeline, and a training time and a training goal corresponding to the least one training stage; generating a voice information according to the training script, and outputting the voice information by an audio outputting unit; receiving a physiological signal sensed by a physiological signal sensor in the at least one training stage, and analyzing the physiological signal utilized an analyzing module to obtain an analysis result; and determining whether the analysis result meets the training goal corresponding to the at least one training stage; if not, outputting a voice reminding by the audio outputting unit.

Another aspect of the disclosure is to provide a training system. The training system includes an electronic device and a physiological signal sensor. The electronic device includes a storage device, a processor and an audio outputting unit. The storage device is configured for storing a training script; wherein the training script includes at least one training stage corresponding to a timeline, and a training time and a training goal corresponding to the least one training stage. The processor is electrically connected to the storage device, and is configured for generating a voice information according to the training script. The audio outputting unit is electrically connected to the processor, and is configured for outputting the voice information. The physiological signal sensor is electrically connected to the electronic device, and is configured for sensing a physiological signal in the at least one training stage, and transmitting the physiological signal to the electronic device. Wherein processor is further configured for analyzing the physiological signal utilized an analyzing module to obtain an analysis result, and determining whether the analysis result meets the training goal corresponding to the at least one training stage; if not, outputting a voice reminding by the audio outputting unit.

Another aspect of the disclosure is to provide a non-transitory computer-readable medium including one or more sequences of instructions to be executed by a processor for performing a training method, wherein the training method includes operations of: receiving a training script; wherein the training script includes at least one training stage corresponding to a timeline, and a training time and a training goal corresponding to the least one training stage; generating a voice information according to the training script, and outputting the voice information by an audio outputting unit; receiving a physiological signal sensed by a physiological signal sensor in the at least one training stage, and analyzing the physiological signal utilized an analyzing module to obtain an analysis result; and determining whether the analysis result meets the training goal corresponding to the at least one training stage; if not, outputting a voice reminding by the audio outputting unit.

Based on aforesaid embodiments, the training method, training system and non-transitory computer-readable medium are capable of generating the training script automatically; providing the virtual coaches interact with the fitness enthusiasts, and monitoring the posture and health conditions of the fitness enthusiasts. In some embodiments, this disclosure is able to generate automatically the training script and interact with the fitness enthusiasts.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIG. 1 is a functional block diagram illustrating a training system according to an embodiment of the disclosure.

FIG. 2 is a functional block diagram illustrating a server according to an embodiment of the disclosure.

FIG. 3 is a flow diagram illustrating a training method according to an embodiment of this disclosure.

FIG. 4 is a schematic diagram illustrating training script according to an embodiment of this disclosure.

FIG. 5 is a flow diagram illustrating a training method according to an embodiment of this disclosure.

FIG. 6 is a flow diagram illustrating a training method according to an embodiment of this disclosure.

### DETAILED DESCRIPTION

It will be understood that, in the description herein and throughout the claims that follow, when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Moreover, “electrically connect” or “connect” can further refer to the interoperation or interaction between two or more elements.

It will be understood that, in the description herein and throughout the claims that follow, although the terms “first,” “second,” etc. may be used to describe various elements, these elements should not be limited by these terms. These terms are only used to distinguish one element from another. For example, a first element could be termed a second element, and, similarly, a second element could be termed a first element, without departing from the scope of the embodiments.

It will be understood that, in the description herein and throughout the claims that follow, the terms “comprise” or “comprising,” “include” or “including,” “have” or “having,” “contain” or “containing” and the like used herein are to be understood to be open-ended, i.e., to mean including but not limited to.

It will be understood that, in the description herein and throughout the claims that follow, the phrase “and/or” includes any and all combinations of one or more of the associated listed items.

It will be understood that, in the description herein and throughout the claims that follow, words indicating direction used in the description of the following embodiments, such as “above,” “below,” “left,” “right,” “front” and “back,” are directions as they relate to the accompanying drawings. Therefore, such words indicating direction are used for illustration and do not limit the present disclosure.

It will be understood that, in the description herein and throughout the claims that follow, unless otherwise defined, all terms (including technical and scientific terms) have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Any element in a claim that does not explicitly state “means for” performing a specified function, or “step for” performing a specific function, is not to be interpreted as a “means” or “step” clause as specified in 35 U.S.C. § 112(f). In particular, the use of “step of” in the claims herein is not intended to invoke the provisions of 35 U.S.C. § 112(f).

Reference is made to FIG. 1, which is a functional block diagram illustrating a training system 100 according to an embodiment of the disclosure. As shown in FIG. 1, the training system 100 includes a server 110, an electronic device 130, and a physiological signal sensor 150. The server 110 and the physiological signal sensor 150 are communicated with the electronic device 130 via the communication network. The electronic device 130 includes a storage device 131, a processor 132, an audio outputting unit 133, an audio receiving unit 134 and a user interface unit 135. The storage device 131 is configured to store a training script. The processor 132 is electrically connected to the storage device 131, and configured to generate a voice information according to the training script. The audio outputting unit 133 is electrically connected to the processor 132, and configured to output the voice information. The audio receiving unit 134 is electrically connected to the processor 132, and configured to receive a user voice. The user interface unit 135 is electrically connected to the processor 132, and configured to provide user to select the training script. Wherein, the training script includes at least one training stage corresponding to a timeline, and a training time and a training goal corresponding to the least one training stage. In some embodiments, the processor 132 is further configured to analyze the physiological signal utilized an analyzing module to obtain an analysis result, and to determine whether the analysis result meets the training goal corresponding to the at least one training stage; if not, outputting a voice reminding by the audio outputting unit 133.

In the embodiments of the disclosure, the electronic device 130 can be implemented by a smart phone, wearable device, a dedicated mobile device, or a controller of a fitness device, etc. The storage device 131 can be implemented by a memory, a hard disk, a flash drive, a memory card, etc. In some embodiments, computer programs and data can be stored on the portable computer readable recording medium, and can be loaded onto the storage device 131 through an I/O interface. The I/O interface may also be connected to a display. The processor 132 can be implemented as an integrated circuit, such as a micro-controller, a microprocessor, a digital signal processor, an application specific integrated circuit (ASIC), a logic circuit or other similar elements, or a combination thereof. The physiological signal sensor 150 can be implemented by a heart rate sensor, an electrocardiogram sensor (ECG), an electromyogram detector (EMG), a motion sensor, an inertial sensor, or an posture sensor.

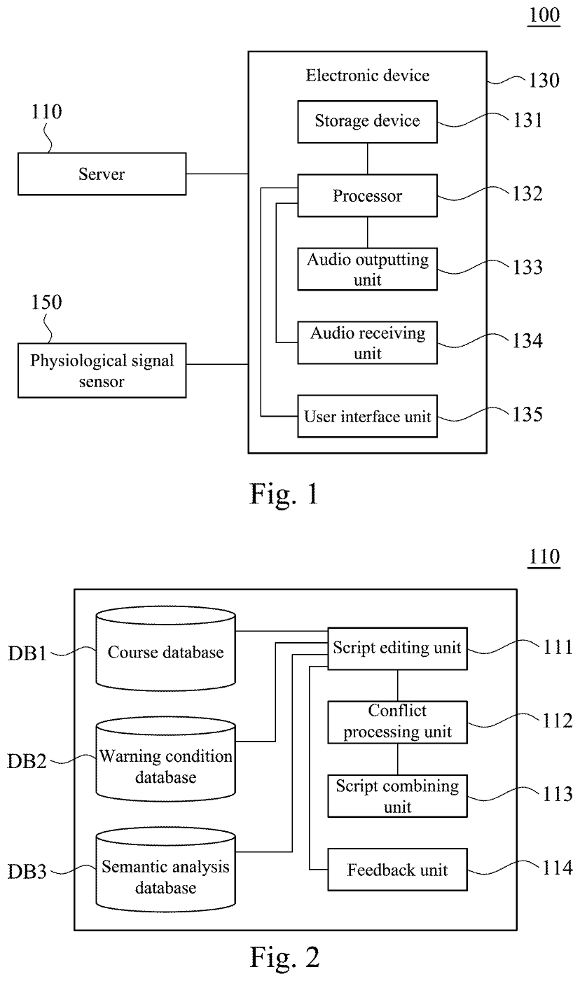

Reference is made to FIG. 2, which is a functional block diagram illustrating a server 110 according to an embodiment of the disclosure. As shown in FIG. 2, the server 110 includes a script editing unit 111, a conflict processing unit 112, a script combining unit 113, and a feedback unit 114. The script editing unit 111 is electrically connected to the conflict processing unit 112, the feedback unit 114, a course database DB1, a warning condition database DB2 and a semantic analysis database DB3. The conflict processing unit 112 is configured to output the training script to the electronic device 130 via a communication network and the electronic device 130 is configured to store the training script in the storage device 131.

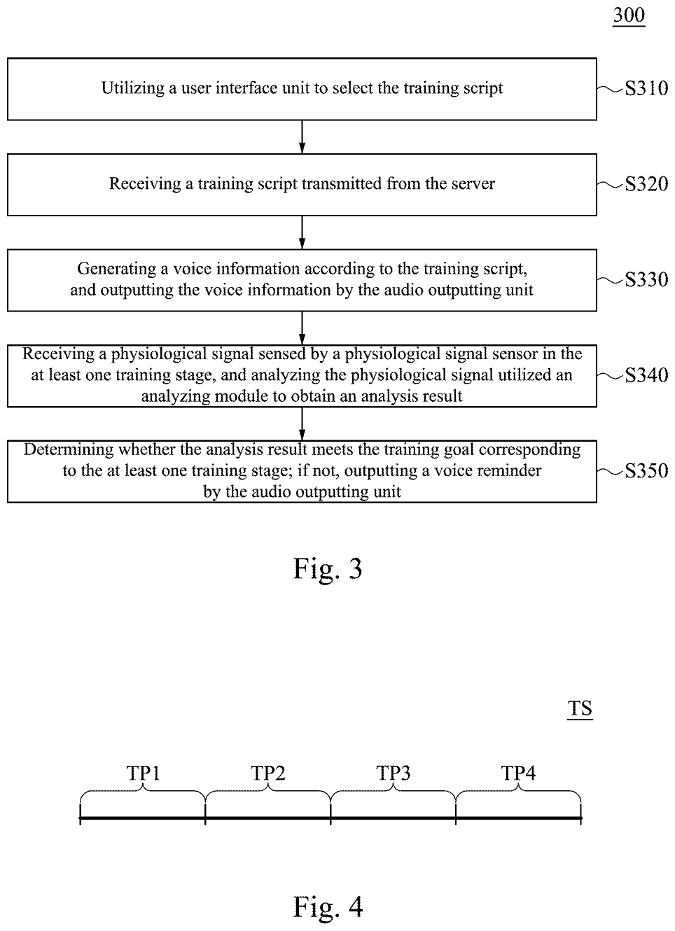

Reference is made to FIG. 3, which is a flow diagram illustrating a training method 300 according to an embodiment of this disclosure. In the embodiment, the training method 300 can be applied to the training system 100 of FIG. 1 and the server 110 of FIG. 2. The server 110 can be utilized to generate the training script and transmit the training script to the electronic device 130. The electronic device 130 can be utilized to remind the user according to the training content of the training script and the health condition of the user. The training script includes the setting information of more than one training stage, and can be executed in the electronic device 130. The training script is continuously executed during the exercising of the fitness enthusiasts according to the training course. The content and target of the training can be explained before the training stage is executed and the instructions that the fitness enthusiasts should perform. The training script can be interacted with the fitness enthusiasts, and monitored posture of the fitness enthusiasts and health condition, etc.

The training method 300 firstly executes step S310 to utilize a user interface unit 135 to select the training script TS. In the embodiment, the user can select a training script via the user interface unit 135 of the electronic device 130. For example, if the user wants to perform a running training, he can choose different running courses (fat burning running, intermittent running, etc.).

Afterwards, the training method 300 executes step S320 to receive a training script TS transmitted from the server 110. In the embodiment, the training script TS includes at least one training stage corresponding to a timeline, and a training time and a training goal corresponding to the least one training stage. Reference is made to FIG. 4, which is a schematic diagram illustrating training script TS according to an embodiment of this disclosure. As shown in FIG. 4, the training script TS includes four training stage TP1˜TP4, and the number of the training stage should not be limited thereto.

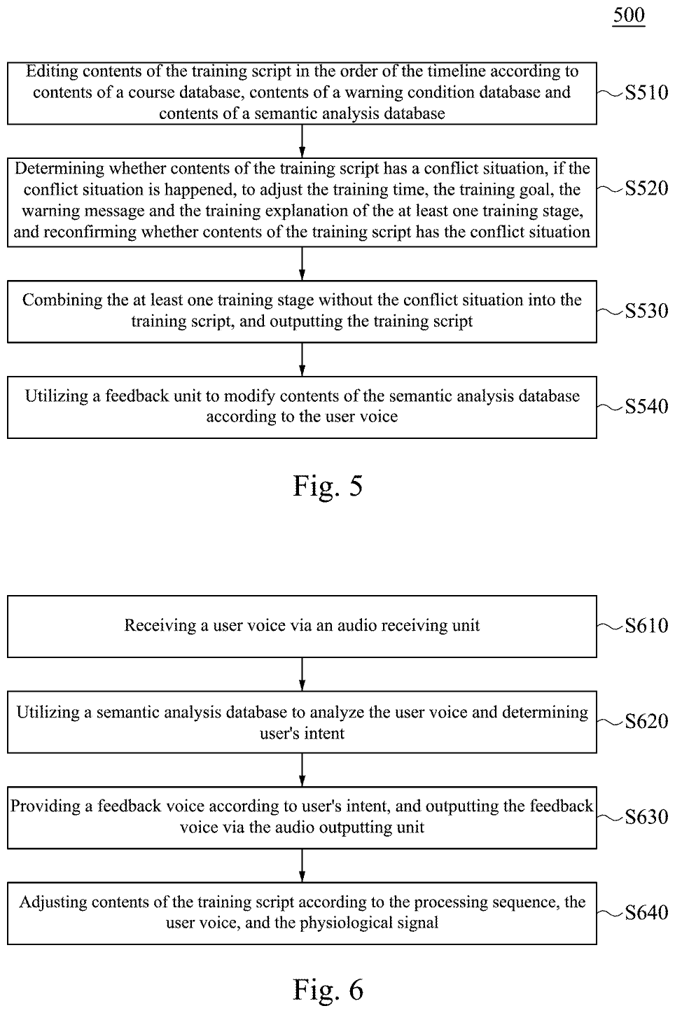

Afterwards, the training script TS is generated by the server 110 before the electronic device 130 receiving the training script TS. The training method 300 further includes steps S510˜S540, reference is made to FIG. 4 and FIG. 5. FIG. 5 is a flow diagram illustrating a training method 300 according to an embodiment of this disclosure. As the embodiment shown in FIG. 5, the training method 300 further executes step S510 to edit contents of the training script TS in the order of the timeline according to contents of a course database DB1, contents of a warning condition database DB2 and contents of a semantic analysis database DB3. In the embodiment, contents of the training script TS includes the training time, the training goal, a warning message and a training explanation of the at least one training stage TP1˜TP4. The training time can be realized as arranging the training time of each training stage TP1˜TP4 according to content of the course database DB1, and the training goal refers to the goals that user needs to achieve in each training stage TP1˜TP4. For example, if the user wants to perform a running training, he can choose different running courses (fat burning running, intermittent running, etc.). If the user selects the intermittent running course, the server 110 can automatically generate a running script based on the user's exercising record. For example, running 5 minutes at X heart rate during training stage TP1, running 10 minutes at Y heart rate during training stage TP2, running 5 minutes at M heart rate during training stage TP3, and running 10 minutes at N heart rate during training stage TP4.

Afterwards, the training explanation refers to the explanation of the course content, the health condition of user, posture reminder of user, or the reminder of the course progress during the training process. For example, it can explain the purpose and effect of intermittent running, reminding the training stage that there are still 5 minutes left or have been run 5 minutes, etc., or reminding the user whether current heart rate has reached the predetermined target or whether the running posture is correct (such as hunchback, whether the arms are naturally swing).

Afterwards, the warning message means that if the user's heart rate exceeds the heart rate threshold of current training stage, a warning message is sent to remind the user that the heart rate is too fast and the exercise intensity needs to be reduced. For example, the target heart rate is 160 bpm, and the heart rate threshold can be 5-10 bpm more than the target heart rate, so that the heart rate threshold is 165 bpm. When the user's heart rate exceeds 165 bpm, the warning message will be send to remind the user reduce the exercise intensity. The warning message also can be generated by the user's running posture, such as improper use of the left and right feet when the user is running, or poor posture, etc. Aforesaid training time, training goal, warning message, and training explanation may output a voice message via the audio output unit 133 in addition to displaying the reminding message via the display on the electronic device 130.

Afterwards, the training method 300 further executes step S520 to determine whether contents of the training script has a conflict situation, if the conflict situation is happened, to adjust the training time, the training goal, the warning message and the training explanation of the at least one training stage TP1˜TP4, and to reconfirm whether contents of the training script has the conflict situation. In the embodiment, the conflict processing unit 112 of the server 110 is configured to arrange contents of the training script TS according to contents of a warning condition database DB2 and the processing sequence. Wherein the processing sequence may be that the training time has a higher priority than the warning message, the warning message has a higher priority than the training explanation, and the training explanation has a higher priority than the training goal.

Afterwards, the conflict situation of contents of the training script TS is means that more than one of content arranged at the same time. For example, the training explanation and the training goal are arranged at the same time in fourth minute of training stage TP1, if there are a lot of messages at the same time, it is difficult for users to read, and therefore, it is necessary to rearrange timing of the training explanation and timing of the training goal according to the priority of processing. In this case, because the training explanation has a higher priority than the training goal, the training explanation can still be arranged in the fourth minute of the training stage TP1, and the training goal need to be arranged after the training explanation.

Afterwards, the training method 300 further executes step S530 to combine the at least one training stage TP1˜TP4 without the conflict situation into the training script TS, and to output the training script TS. In the embodiment, each training stage TP1˜TP4 is determined whether the training time, the training goal, the warning message, and the training explanation has a conflict situation at the same time. If the conflict situation is happened, it is need to readjust timing of the training time, the training goal, the warning message, and the training explanation. When the conflict situation is processed, the training stages TP1˜TP4 can be combined into the training script TS and transmitted to the electronic device 130.

Afterwards, the training method 300 further executes step S540 to utilize a feedback unit to modify contents of the semantic analysis database DB3 according to the user voice. In the embodiment, when the electronic device 130 is configured to receive the user voice stored in the format of event logs, the user voice is transmitted to the server 110. Therefore, the server 110 is configured to modify contents of the semantic analysis database DB3 according to the event logs file of the user voice.

Afterwards, the training method 300 further executes step S330 to generate a voice information according to the training script TS, and output the voice information by the audio outputting unit 133. In the embodiment, after receiving the training script TS, the processor 132 of the electronic device 130 is configured to analyze the training time, the training goal, the warning message, and the training explanation in the training script TS. The training time, the training goal, the warning message, and the training explanation are transformed into the voice message and the processor 132 is configured to control the audio outputting unit 133 to output the voice message.

Afterwards, the training method 300 further executes step S340 to receive a physiological signal sensed by a physiological signal sensor in the at least one training stage TP1˜TP4, and analyze the physiological signal utilized an analyzing module to obtain an analysis result. In the embodiment, the physiological signal sensor 150 can be implemented by a heart rate sensor or a posture sensor. The user can wear the heart rate sensor or the posture sensor to monitor the user's blood pressure, the heart rate and the posture during exercising. The analyzing module is configured to record the user's blood pressure, heart rate, or the angle of body and trunk during exercising. The processor 132 is configured to determine the health condition of the user according to the analysis module during exercising.

Afterwards, the training method 300 further executes step S350 to determine whether the analysis result meets the training goal corresponding to the at least one training stage TP1˜TP4; if not, output a voice reminding by the audio outputting unit 133. For example, the training goal is to run for 10 minutes and the heart rate value needs to reach 140 bpm in the training stage TP2, if the user's heart rate value is less than or larger than a certain range, the audio outputting unit 133 is configured to send a voice reminding or an alert voice. When the server 110 generated the training script TS, the alert voice is a present warning message, and the processor 132 is configured to transform the present warning message into the alert voice outputted by the audio outputting unit 133. The certain range can be realized as normal heart rate range suitable for the user, for example, it is assumed that the heart rate threshold is 160 bpm, if user's heart rate exceeds 160 bpm, the audio outputting unit 133 is configured to send the alert voice to remind user to slow the heartbeat. If user's heart rate is less than 140 bpm (it means user do not reach the training goal), the voice reminding is sent to remind the user that there is still a gap between the current heart rate and the target heart rate, and it is necessary to increase the exercise intensity.

Afterwards, because the user can interact with the electronic device 130 at any time, the processor 132 is configured to receive the user's voice through the audio receiving unit 134. Reference is made to FIG. 6. FIG. 6 is a flow diagram illustrating a training method 300 according to an embodiment of this disclosure. The training method 300 further executes step S610 to receive a user voice via an audio receiving unit 134. In the embodiment, user can trigger the processor 132 in a specific way. For example, when the user is exercising, the processor 132 may have an environmental noise problem, and thus the processor 132 may not continuously receive the audio signal via the audio receiving unit 134. Therefore, the user can transmit a trigger signal to indicate that the user will start to speak. The trigger signal can be set by touch, pressing, multi-finger touch and the trigger signal should not be limited thereto.

Afterward, training method 300 further executes step S620 to utilize a semantic analysis database DB3 to analyze the user voice and to determine user's intent. In the embodiment, when the processor 132 is configured to receive the user voice via the audio receiving unit 134, the processor 132 can analyze user's intention utilized a pre-stored semantic parsing database DB3. For example, if the user provides a question “how long have I been running?”, the user's intention analyzed by the processor 132 is user want to know the duration of the exercise.

Afterward, training method 300 further executes step S630 to provide a feedback voice according to user's intent, and to output the feedback voice via the audio outputting unit 133. Based on aforesaid embodiment, after analyzing the user's intention (the duration of the exercise), the processor 132 may output the feedback voice about the exercise time in the semantic analysis database DB3 via the audio outputting unit 133.

Afterward, training method 300 further executes step S640 to adjust contents of the training script TS according to the processing sequence, the user voice, and the physiological signal. In the embodiment, the processing sequence may be that the training time has a higher priority than the warning message, the warning message has a higher priority than the training explanation, and the training explanation has a higher priority than the training goal. Therefore, when the processor 132 receives the user voice or the physiological signal, the processor 132 is configured to determine whether the user's physiological signal exceeds the heart rate threshold in the current training stage, if the user's physiological signal (user's heart rate) exceeds the heart rate threshold, the processor 132 is configured to insert the warning message (message warning the user that the heart rate exceeds the heart rate threshold) into the current training script TS and execute the warning message in the highest priority order.

Afterwards, in another embodiment, if the user provides the question “how long have I been running?”, after the analysis of user's intention of aforesaid steps S610˜S630, the processor 132 can output the feedback voice. The feedback voice has a lower priority than the warning message, but the feedback voice has a higher priority than the training explanation and the training goal. Therefore, if the warning message and the feedback voice are not happened in the same time, the feedback voice has the highest priority order. The processor 132 is configured to insert the feedback voice into the current training script TS and execute the feedback voice in the highest priority order. If the warning message and the feedback voice are happened in the same time, the processor 132 preferentially outputs the warning message through the audio outputting unit 133, and then outputs the feedback voice.

Based on aforesaid embodiments, the training method, training system and non-transitory computer-readable medium are capable of generating the training script automatically; providing the virtual coaches interact with the fitness enthusiasts, and monitoring the posture and health conditions of the fitness enthusiasts. In some embodiments, this disclosure is able to generate automatically the training script and interact with the fitness enthusiasts.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A training method, applied to an electronic device and a server, wherein the training method comprising:
receiving a training script; wherein the training script includes at least one training stage corresponding to a timeline, and a training time and a training goal corresponding to the least one training stage;
generating a voice information according to the training script, and outputting the voice information by an audio outputting unit;
receiving a physiological signal sensed by a physiological signal sensor in the at least one training stage, and analyzing the physiological signal by utilizing an analyzing module to obtain an analysis result; and
determining whether the analysis result meets the training goal corresponding to the at least one training stage; if not, outputting a voice reminding by the audio outputting unit.

2. The training method of claim 1, wherein the electronic device is configured for executing following steps:
receiving a user voice via an audio receiving unit;
utilizing a semantic analysis database to analyze the user voice and determining user's intent; and
providing a feedback voice according to user's intent, and outputting the feedback voice via the audio outputting unit.

3. The training method of claim 1, wherein the server is configured for executing following steps:
editing contents of the training script in the order of the timeline according to contents of a course database, contents of a warning condition database and contents of a semantic analysis database; wherein contents of the training script comprises the training time, the training goal, a warning message and a training explanation of the at least one training stage;
determining whether contents of the training script has a conflict situation, if the conflict situation is happened, adjusting the training time, the training goal, the warning message and the training explanation of the at least one training stage, and reconfirming whether contents of the training script has the conflict situation; and
combining the at least one training stage without the conflict situation into the training script, and outputting the training script.

4. The training method of claim 3, wherein contents of the training script is arranged according to a processing sequence; wherein the processing sequence is sequentially the training time, the warning message, the training explanation, and the training goal.

5. The training method of claim 4, the electronic device is configured for executing following step:
adjusting contents of the training script according to the processing sequence, the user voice, and the physiological signal.

6. The training method of claim 3, the server is configured for executing following step:
utilizing a feedback unit to modify contents of the semantic analysis database according to the user voice.

7. The training method of claim 1, wherein receiving the physiological signal sensed by the physiological signal sensor, and analyzing the physiological signal utilized the analyzing module comprises:
detecting a heart rate physiological signal by a heart rate sensor, and utilizing the analyzing module to the heart rate physiological signal; and
detecting a posture physiological signal by a posture sensor, and utilizing the analyzing module to analyze the posture physiological signal.

8. The training method of claim 1, further comprising:
utilizing a user interface unit to select the training script.

9. A training system, comprising:
an electronic device, comprises:
a storage device, configured for storing a training script; wherein the training script includes at least one training stage corresponding to a timeline, and a training time and a training goal corresponding to the least one training stage;
a processor is electrically connected to the storage device, and configured for generating a voice information according to the training script; and
an audio outputting unit is electrically connected to the processor, and configured for outputting the voice information; and
a physiological signal sensor, is electrically connected to the electronic device, and configured for sensing a physiological signal in the at least one training stage, and transmitting the physiological signal to the electronic device;
wherein processor is further configured for analyzing the physiological signal by utilizing an analyzing module to obtain an analysis result, and determining whether the analysis result meets the training goal corresponding to the at least one training stage; if not, outputting a voice reminding by the audio outputting unit.

10. The training system of claim 9, wherein electronic device further comprising:
an audio receiving unit is electrically connected to the processor, and configured for receiving a user voice;
wherein, the storage device is further configured for storing a semantic analysis database; the processor is further configured for utilizing the semantic analysis database to analyze and determine the user voice and intent of the user voice, and providing a feedback voice according to intent of the user voice; the audio outputting unit is further configured for outputting the feedback voice.

11. The training system of claim 9, wherein training system further comprises a server, communicated with the electronic device via a communication network, and is configured for outputting the training script, the server further comprising:
a script editing unit is configured for editing contents of the training script in the order of the timeline according to contents of a course database, contents of a warning condition database and contents of a semantic analysis database;
wherein contents of the training script comprises the training time, the training goal, a warning message and a training explanation of the at least one training stage;
a conflict processing unit, is electrically connected to the script editing unit, and configured for determining whether contents of the training script has a conflict situation, if the conflict situation is happened, adjusting the training time, the training goal, the warning message and the training explanation of the at least one training stage, and reconfirming whether contents of the training script has the conflict situation; and;
a script combining unit, is electrically connected to the conflict processing unit, and configured for combining the at least one training stage without the conflict situation into the training script, and outputting the training script to the electronic device.

12. The training system of claim 11, wherein contents of the training script is arranged according to a processing sequence; wherein the processing sequence is sequentially the training time, the warning message, the training explanation, and the training goal.

13. The training system of claim 12, wherein the electronic device is further configured for adjusting contents of the training script according to the processing sequence, the user voice, and the physiological signal.

14. The training system of claim 11, wherein the server further comprising:
a feedback unit, is electrically connected to the script editing unit, and configured for modifying contents of the semantic analysis database according to the user voice.

15. The training system of claim 9, wherein the physiological signal sensor comprises:
a heart rate sensor, configured for detecting a heart rate physiological signal, and utilizing the analyzing module to the heart rate physiological signal; and
a posture sensor, configured for detecting a posture physiological signal, and utilizing the analyzing module to analyze the posture physiological signal.

16. The training system of claim 9, wherein the electronic device further comprising:
a user interface unit, is electrically connected to the processor, and configured for providing user to select the training script.

17. A non-transitory computer-readable medium including one or more sequences of instructions to be executed by a processor for performing a training method, wherein the method comprises:
receiving a training script; wherein the training script includes at least one training stage corresponding to a timeline, and a training time and a training goal corresponding to the least one training stage;
generating a voice information according to the training script, and outputting the voice information by an audio outputting unit;
receiving a physiological signal sensed by a physiological signal sensor in the at least one training stage, and analyzing the physiological signal utilized an analyzing module to obtain an analysis result; and
determining whether the analysis result meets the training goal corresponding to the at least one training stage; if not, outputting a voice reminding by the audio outputting unit.
